(19) United States
(12) Patent Application Publication
(10) Pub. No.: US 2007/0135377 A1
(43) Pub. Date: Jun. 14, 2007

(54) Thixotropic anti-viral formulation

(76) Inventors: Stephen B. Roman, Phoenix, AZ (US); Gary S. Kehoe, Phoenix, AZ (US)

Correspondence Address: TOD R NISSLE, PO BOX 55630, PHOENIX, AZ, 85078, US

(21) Appl. No.: 11/704,566

(22) Filed: Feb. 9, 2007

Related U.S. Application Data
(63) Continuation-in-part of application No. 11/501,609, filed on Aug. 9, 2006.
Application No. 11/501,609 is a continuation-in-part of application No. 11/210,293, filed on Aug. 24, 2005.
(60) Provisional application No. 60/635,095, filed on Dec. 10, 2004.
(60) Provisional application No. 60/772,448, filed on Feb. 10, 2006.

(51) Int. Cl. A61K 31/715

(52) U.S. Cl. 514/54

(57) ABSTRACT

A thixotropic anti-viral formulation includes a high molecular weight, thixotropic, gel-forming, naturally occurring polysaccharide extracted from algae and comprised of repeating sulfated and non-sulfated galactose and 3,6 anhydrogalactose (3,6-AG) units, and includes water.

This application is a continuation-in-part of application Ser. No. 11/501,609, filed Aug. 9, 2006, which is a continuation-in-part of application Ser. No. 11/210,293 filed Aug. 24, 2005 and also derives from and claims priority based upon provisional patent application Ser. No. 60/635,095 filed Dec. 10,2004 and provisional patent application Ser. No. 60/772,448, filed Feb. 10, 2006.

This invention pertains to lubricants other formulations for epithelial tissue layers, especially mucosal tissue, which formulations can provide high lubricity, provide protection from disease, deliver medicaments, and exhibit a viscosal stability sufficient to maintain the personal lubricants in contact with mucousal tissue for extended periods of time. The invention also pertains to methods of preparing, dispensing and using such formulations in accordance with the invention.

More particularly, the invention pertains to formulations to protect epithelial tissues from contact with and penetration by viruses.

Conventional formulations, once applied to epithelial tissues, often tend to have little viscosity, tend to lose their viscosity and travel over and away from desired epithelial tissues at a faster than desired rate, or, in the manner of ZICAM (TM) gel tend to dry up in the nasal cavity, to leave behind a cellulose mass, and to not coat and maintain contact with substantially all desired areas in the throat as well as in the nose. Consequently, it is difficult to apply such formulations and to have the formulations remain in position for an extended period of time. In addition, such formulations can contain artificial compositions, can permit movement of macrophages through the lubricant, and can be inconvenient to apply.

It would be desirable to provide a formulation that would maintain contact with epithelial tissues for an extended period of time, that would travel from the nasal cavity into desired areas of the throat, that would not dry up and would remain moist, that inhibit the passage of macrophages and other harmful bodies through the formulation, that would not contain artificial compositions, that would be convenient to apply, and that would permit shear induced movement of formulation to facilitate travel of the formulation over an extended epithelial area.

We have discovered an improved anti-viral formulation. The formulation comprises a thixotropic gel having a viscosity in the range of 1,000 to 80,000 centipoise; having a pH in the range of 2.6 to 10; and, comprising naturally occurring compositions. The formulation preferably includes natural components including a high molecular weight, thixotropic, gel-forming, naturally-occurring polysaccharide extracted from algae and composed of repeating sulfated and non-sulfated galactose and 3,6 anhydrogalactose (3,6-AG) units; and, water. The formulation inhibits the passage of macrophages through the composition, is natural, and retains moisture. Formulations prepared in accordance with the invention can be utilized when the formulations are room temperature, when the formulations are chilled, or when the formulations are frozen.

One embodiment of the invention comprises a method of protecting mucosal tissue to prevent Rhino virus from entering or adhering to ICAM-1 and ICAM-2 sites. The method comprises the step of providing a container of a personal lubricant thixotropic formulation to dispense multiple equivalent doses of the formulation. The formulation has a viscosity in the range of 200 to 80,000 centipoise, preferably 1,000 to 30,000 centipoise, and has a pH in the range of 2.6 to 10, and comprises water and a high molecular weight, thixotropic, gel-forming, naturally-occurring polysaccharide extracted from algae and composed of repeating sulfated and non-sulfated galactose and 3,6 anhydrogalactose (3,6-AG) units. The method also includes the steps of applying with the container at least one dose of the formulation to muscosal tissue in an individual's nose; and, allowing shear produced by movement of the nose to reduce the viscosity of the thixotropic formulation and facilitate movement of some of the formulation from the nose to the individual's throat.

The particular polysaccharide presently utilized in the practice of the invention is critical. Although a multitude of polysaccharides exist, the critical polysaccharide utilized in the invention is a high molecular weight thixotropic polysaccharide made up of repeating galactose and 3,6 anydrogalactose (3,6-AG) units, both sulfated and nonsulfated and extracted from algae, typically Eucheuma, Chondrus, and Gigartina red benthic marine algae that are multicellular and macrothallic. As used herein, a polysaccharide is thixotropic when it produces a thixotropic solution or gel when admixed with water or another liquid.

Three specific types of high molecular weight galactose polysaccharides extracted from marine algae are kappa, iota, and lambda.

Kappa polysaccharide typically forms a strong, rigid aqueous gel; has some syneresis; and, forms a helix with potassium ions. Calcium ions cause the helices in kappa formed gel to aggregate and cause the gel to contract and become brittle. Gel formed with kappa polysaccharide is slightly opaque, but becomes clear when sugar is added. Kappa polysaccharide is about 25% ester sulfate and about 34% 3,6-AG.

Iota polysaccharide forms an elastic aqueous thixotropic gel and forms a helix with calcium ions. Limited aggregation in iota formed gel contributes to the elasticity of the gel. There is no syneresis. The gel is clear. When iota formed gel is frozen and thawed, its viscosity remains stable, as generally do gels formed with iota polysaccharide in combination with kappa polysaccharide and/or lambda polysaccharide. Iota polysaccharide is about 32% ester sulfate and 30% 3, 6-AG.

Lambda polysaccharide does not form an aqueous gel. Lambda polysaccharide is about 35% ester sulfate and includes little or no 3,6-AG.

While lambda and kappa polysaccharide can be utilized alone, in combination with each other, or in combination with iota polysaccharide in producing formulations utilized in the invention, when a thixotropic formulation is desired-which is the case in the presently preferred embodiment of the invention-iota polysaccharide must be utilized. Iota polysaccharide presently comprises at least 25%, preferably at least 33%, most preferably at least 50% of a quantity of high molecular weight galactose algae polysaccharide utilized to prepare a batch or quantity of the formulation in accordance with the invention. The remaining portion of the quantity of galactose algae polysaccharide used to prepare a batch of the formulation can comprise lambda or kappa polysaccharide. When solids are admixed with water to produce the formulation, the high molecular weight galactose algae polysaccharide comprises at least 50%, preferably at least 75%, most preferably at least 80% of the solids, while water or other liquids comprise the remainder of the composition.

The concentration of high molecular weight galactose polysaccharide in the formulation of the invention is in the range of 0.1% to 5.0% by weight, preferably in the range of 1% to 4% by weight, most preferably in the range of 1.5% to 3.5% by weight. As noted, the galactose polysaccharide can consist of iota, lambda, and/or kappa polysaccharide.

If the formulation of the invention includes 0.5% by weight of iota galactose polysaccharide, thixotropic properties are not apparent. If the formulation includes 0.75% by weight iota polysaccharide, some thixotropic properties are evidenced. 1.0% by weight of iota polysaccharide provides more evidence of thixotropic properties; 1.5% by weight provides good evidence; and, when there is 1.75% by weight iota polysaccharide the thixotropic property of the gel formulation is very noticeable. Consequently, it is preferred that the formulation of the invention include at least 1.0% by weight iota polysaccharide, preferably at least 1.5% by weight iota polysaccharide, and most preferably at least 1.75% by weight iota polysaccharide. Lesser fractions of lambda and kappa polysaccharides are normally, but not necessarily, included with iota polysaccharide.

Iota, kappa, and lambda polysaccharides are sold by various sources, including FMC Corporation, 1735 Market Street, Philadelphia, Pa. 19103, and CP Kelco, 311 S, Wacker Drive, Suite 3700, Chicago, Ill. 60606. Examples of galactose polysaccharides sold by FMC Corporation are 373/Gelcarin GP 911 [Kappa polysaccharides comprise at least majority of composition], 335/Gelcarin GP 379 [Iota polysaccharides comprise at least majority of composition], 303/Gelcarin GP 812 [Kappa polysaccharides], 205Niscarin GP 109 [Lambda polysaccharides], 201/Viscarin GP 209 [Lambda polysaccharides], and, 357/Seaspen PF [Iota polysaccharides, phosphates, CaSO4-2H20]. Examples of galactose polysaccharides sold by CP Kelco are Genuvisco type X-931-03 (CP Kelco), and Genuvisco type X-923-03 (CP Kelco) [Iota polysaccharides].

Glycerin can be included in the anti-viral formulation of the invention as an emollient and to slow the evaporation of moisture from the formulation. Glycerin is a naturally occurring substance and can comprise from 0.5% to 12.5% by weight of the formulation. It is presently preferred to incorporate from 2.0% to 8.0% by weight glycerin in the formulation. Propylene glycol or any other conventional desired emollients can be utilized in the formulation.

Minor effective amounts of preservatives, typically in the range of 0.01% to 1.5% by weight, can be included in the formulation. By way of example, and not limitation, methylparaben, propylparaben, potassium sorbate, and benzoic acid are common preservatives than can be utilized.

Effective amounts of appropriate acidic or basic compositions can be include in the formulation to adjust and control pH in the desired range of 2.6 to 10. The presently preferred pH is 5.5. By way of example, and not limitation, citric acid and sodium hydroxide comprise compositions commonly utilized to adjust the pH of the personal lubricant.

Minor effective amounts of flavoring, topical stimulants (i.e., to produce a warming or cooling sensation) coloring, or odor producing compositions (i.e., scents) can be incorporated in the formulation in either a liquid, solid or gaseous form or mixture thereof.

The water utilized preparing the personal lubricant can be de-ionized water, USP water, de-chlorinated water, mineral water, water treated with activated carbon, tap water, etc. Naturally occurring oils or other fluids can, if desired, be utilized in place of or in combination with water.

Dosage can vary per the user's discretion, but the volume of a single dose typically is in the range of 0.05 mL (milliliter) to 4 mL, preferably 0.10 mL to 0.75 mL.

The following examples are given by way of illustration and not limitation of the invention.

### EXAMPLE I

The following ingredients are provided.

| Ingredient | Weight Percent |
|---|---|
| Polysaccharide | 4.5 |
| Iota polysaccharide | 3.0 |
| Kappa polysaccharide | 0.75 |
| Lambda polysaccharide | 0.75 |
| Sodium Hydroxide (pH adjustment) | .05 |
| Citric Acid (pH adjustment) | .05 |
| Methylparaben (preservative) | .10 |
| Propylparaben (preservative) | .10 |
| Glycerin | 1.00 |
| De-ionized water | 94.2 |

The water, polysaccharides, sodium hydroxide, and glycerin are admixed under agitation to the water at room temperature. The pH of the resulting aqueous formulation is adjusted to 4.0 by adding the citric acid. The preservatives are added while the aqueous formulation is stirred. Care is taken to avoid entrainment of air when the aqueous formulation is stirred or otherwise agitated. The resulting anti-viral gel formulation has a viscosity of 5,500 centipoise. The viscosity of formulations in accordance with the invention is, as noted, in the range of 200 to 80,000 cps, but a viscosity in the range of 1,000 to 30,000 cps is preferred. A quantity of the anti-viral formulation is charged in a container that permits a selected metered amount of the formulation to be dispensed from the container on multiple occasions. The container selected is configured to dispense 0.5 mL of the formulation each time the container is utilized. The container can be operated to deliver the exact amount of formulation (dosage) prescribed. Once such container is produced by Mega Pumps L.P. of 611 Industrial Way West, Eatontown, N.J. 07724. Any desired container can be utilized to dispense a metered amount of the formulation. A particular desired feature of a container produced by Mega Pumps is that the container can dispense formulation when the container is in any orientation. Further, the container prevents the admixing of air with the formulation when the gel is dispensed. The container is preferably designed such that the container can--preferably with only a single hand--be grasped and manipulated by a user to dispense a dose of formulation from a nozzle on the container directly into a user's nose. The nozzle can be configured to be inserted a selected distance in a user's nose, etc. prior to operating the container to select a metered dose of formulation. Movement of the nose, and therefore the formulation in the nose, generates shear acting on the formulation and causes the formulation to liquify and spread out over the contact surface areas in both the nose and throat.

### EXAMPLE II

Example I is repeated except that instead of 3% by weight of iota polysaccharide and 0.75% of lambda polysaccharide being utilized, 1.5% by weight of iota polysaccharide is utilized and 1% by weight of lambda polysaccharide is utilized. Similar results are obtained.

### EXAMPLE III

Example I is repeated except that instead of 3% by weight of iota polysaccharide, 0.75% by weight of iota polysaccharide, and 0.75% by weight of kappa polysaccharide being utilized, 3.0% by weight of iota polysaccharide is utilized. Similar results are obtained.

### EXAMPLE IV

Example I is repeated except that, 0.65% of sodium hydroxide is utilized to adjust the pH to 8.0 instead of 5.5. Similar results are obtained.

### EXAMPLE V

0.5 mL of the formulation of EXAMPLE I is dispensed into the nose of a woman. Shear produced by movement of the individual's nose causes the formulation to coat epithelial tissues in the nose and throat of the individual.

The iota, kappa, and lambda polysaccharides are sensitive to cations like sodium, potassium, and magnesium. The sodium cation increases the viscosity of the polysaccharides. It is therefore, in one embodiment of the invention, preferred to utilize a composition or component that functions as a fragrance and preservative and that includes a sodium, potassium, and/or magnesium ion. The concentration of the fragrance-preservative-ion composition in the finished product is in the range of 0.01% to 3.0% by weight. One such preferred composition comprises sodium phytate.

The glycerin that is preferably utilized is 97.5% glycerin, with the remainder of the glycerin being water. Some “glycerin” compositions are 60% to 70% by weight glycerin, with the remainder being water. Using glycerin compositions that are only 60% to 70% glycerin, although possible, is not presently preferred because the polysaccharides will tend to clump into balls when they contact the water in the glycerin. Glycerin compositions that are at least 90% by weight glycerin are preferred in the practice of the invention because the polysaccharides tend during mixing to disperse uniformly in such glycerin compositions and tend not to clump.

Having described our invention in such terms as to enable those of skill in the art to make and practice it, and having described the presently preferred embodiments thereof, We claim:

1. A method of coating mucosal tissue in the body to inhibit a virus from contacting ICAM-1 sites in the tissue, comprising the steps of
(a) providing a high molecular weight, thixotropic, gel-forming, naturally-occurring polysaccharide extracted from algae and composed of repeating sulfated and un-sulfated galactose and 3,6 anydrogalactose (3,6-AG) units;
(b) providing water;
(c) forming a anti-viral formulation by admixing said water and polysaccharide, said formulation having a viscosity in the range of 200 to 45,000 centipoise, and having a pH in the range of 2.6 to 10;
(d) placing in a container a selected quantity of said formulation to dispense multiple doses of said formulation;
(e) applying to mucosal tissue in an individual's nose with said container said formulation; and,
(f) coating mucosal tissue in the individual's throat when movement of the individual's nose produces shear that reduces the viscosity of the formulation to facilitate movement of some of the formulation over mucosal tissue in the individual's throat.

2. A thixotropic anti-viral composition to prevent Rhino virus from passing through the composition and entering or adhering to ICAM-1 sites in epithelial tissue, comprising a high molecular weight, thixotropic, gel-forming, naturally-occurring polysaccharide extracted from algae and composed of repeating sulfated and non-sulfated galactose and 3,6 anhydrogalactose (3,6-AG) units; and water, said composition having a viscosity in the range of 200 to 45,000 centipoise and a pH in the range of 2.6 to 10.
